(19) United States
(12) Patent Application Publication
(10) Pub. No.: US 2020/0308559 A1
(43) Pub. Date: Oct. 1, 2020

(54) Composition for detecting protein-protein interactions comprising fragments of secreted alkaline phosphatase (SEAP) and method for detecting protein-protein interactions using the same

(71) Applicant: Tae Uk KIM, Seoul (KR)

(72) Inventor: Tae Uk KIM, Seoul (KR)

(21) Appl. No.: 16/694,490

(22) Filed: Nov. 25, 2019

(30) Foreign Application Priority Data
Mar. 28, 2019 (KR) 10-2019-0035771

(51) Int. Cl. C12N 9/16; C12Q 1/44; C12N 15/85; G01N 33/50

(52) U.S. Cl. CPC C12N 9/16; G01N 33/5008; C12N 15/85; C12Q 1/44

(57) ABSTRACT

Provided are a composition for detecting protein-protein interactions comprising fragments of secreted alkaline phosphatase (SEAP) and a method for detecting protein-protein interactions using the same. According to the composition or the method of the present invention, it is possible to simply detect the protein-protein interactions in the cells without environmental changes (e.g., cell destruction) in the cells. Furthermore, the composition or the method of the present invention can also be used for detection of materials that enhance or inhibit protein-protein interactions.

This application claims priority to Korean Application No. 10-2019-0035771, filed Mar. 28, 2019. The entire text of the above referenced disclosure is specifically incorporated herein by reference.

### TECHNICAL FIELD

The present invention relates to a composition for detecting protein-protein interactions comprising fragments of secreted alkaline phosphatase (SEAP) and a method for detecting protein-protein interactions using the same.

### BACKGROUND ART

Cells perform various biological functions such as gene expression, cell growth, the cell cycle, metabolism, and signaling through various and complex protein-protein interactions to maintain the phenomenon of life. Accordingly, understanding protein-protein interactions in cells and the functions of these interactions is fundamental to understanding the phenomenon of life, and forms an important basis for disease treatment and the development of new drugs.

Existing techniques for examining protein-protein interactions in vitro or in vivo include affinity chromatography, coimmunoprecipitation, phage display, two-hybrid assays, GST fusion protein pulldown assay, immunohistochemistry, etc. These existing techniques have various advantages, but are disadvantageous in detecting protein-protein interactions in the cells rapidly.

Protein affinity chromatography has a disadvantage in that purified proteins must be prepared, and since the protein-protein interactions are confirmed in vitro, a false-positive result may be derived in which proteins which do not interact with each other in the cells may appear to bind due to electrostatic interaction while passing through a column.

Coimmunoprecipitation requires purified, highly sensitive antibodies, and the antibodies need to recognize forms of proteins existing in the cells. Therefore, when the sensitivity and specificity of the antibodies are low, it is difficult to detect protein-protein interactions.

In phage display, since the protein is expressed in a form fused with a capsid or outer protein of a phage, the size of the protein that may be expressed is limited. Many proteins in mammalian cells undergo various modifications after the translation process, but in phages, the proteins do not undergo the same folding and modification after translation as those made in eukaryotic cells, and thus it is difficult to study the modification of the proteins.

Two-hybrid assays have mainly been used in yeast and mammalian cells, and in yeasts, bait proteins are made identically to those in eukaryotic cells, and folding and modification should occur. In the case of two-hybrid assays using mammalian cells, after the synthesis of proteins, folding or modification occurs properly, but since protein-protein interactions are confirmed through transcription activation in the nucleus using a DNA binding domain, in the case of interactions between proteins that interact with each other in the cytoplasm, it is difficult to confirm the interactions in the cytoplasm. Further, when a reporter gene is not sufficiently activated by the protein-protein interactions, there is not a large difference in the activation degree of a control group even when the transcription is instead inhibited by the interactions, and thus it is difficult to detect the interactions.

Immunohistochemistry involves undergoing a process of fixing a sample with paraffin and formalin during preparation of the sample, and during this process, the cells may be affected, and a sensitive antibody is required. After only positions in cells where bait proteins are present are stained with dye, the protein-protein interactions are determined by the positions of the proteins based on these results, and as a result, it is difficult to determine precise protein-protein interactions.

In GST pulldown assays, a process of expressing and purifying bait proteins in bacteria is undergone, but a process of expressing the proteins water-solubly is not easy, and the expressed proteins may also have different structures from proteins expressed in mammalian cells. In addition, since decomposition of the proteins may occur during and after purification, a continuous protein state should be monitored. Further, the binding between the proteins is greatly affected by the composition of the buffer used. Therefore, the GST pulldown assay must be accompanied by research on a suitable buffer composition, and since it is an in vitro experiment, the result obtained may be different from the interactions in vivo.

By overcoming the disadvantages of existing methods for analyzing protein-protein interactions, that is, problems such as the need for purified antigen-specific antibodies, pollution and changes in the cell environment during processes such as cell destruction in the process of performing experiments, and difficulty in protein purification, a new method for detecting protein-protein interactions simply and precisely is required.

With this background, the present inventors made many efforts to detect protein-protein interactions in cells simply and precisely, and as a result, confirmed that by using a fusion protein obtained by fusing a bait (or prey) protein to a fragment of a SEAP protein, the protein-protein interactions may be detected by way of a simple method without performing a cell destruction process, thereby completing the present invention.

### Non-Patent Documents

(Non-Patent Document 1) Gavin et al., Nature 2002, 415:141-147
(Non-Patent Document 2) Ho et al., Nature 2002, 415:180-183
(Non-Patent Document 3) Krogan et al., Nature 2006, 440:637-643

### DISCLOSURE

### Technical Problem

An object of the present invention is to provide a composition for detecting protein-protein interactions comprising: a first construct comprising a polynucleotide encoding a first fusion protein comprising a bait protein and a secreted alkaline phosphatase (SEAP) first fragment protein; and a second construct comprising a polynucleotide encoding a second fusion protein comprising a prey protein and a SEAP second fragment protein.

Another object of the present invention is to provide a method for detecting protein-protein interactions comprising: (a) introducing to cells a first construct comprising a polynucleotide encoding a first fusion protein comprising a bait protein and a SEAP first fragment protein; and a second construct comprising a polynucleotide encoding a second fusion protein comprising a prey protein and a SEAP second fragment protein; (b) expressing the fusion proteins and inducing the protein-protein interactions; and (c) measuring SEAP activities before and after inducing the interactions.

Yet another object of the present invention is to provide a composition for screening a therapeutic agent comprising: a first construct comprising a polynucleotide encoding a first fusion protein comprising a bait protein and a secreted alkaline phosphatase (SEAP) first fragment protein; and a second construct comprising a polynucleotide encoding a second fusion protein comprising a prey protein and a SEAP second fragment protein.

Still another object of the present invention is to provide a composition for detecting a promoter or inhibitor for protein-protein interactions comprising: a first construct comprising a polynucleotide encoding a first fusion protein comprising a bait protein and a SEAP first fragment protein; and a second construct comprising a polynucleotide encoding a second fusion protein comprising a prey protein and a SEAP second fragment protein.

### Technical Solution

Each description and embodiment disclosed in the present invention can also be applied to each other description and embodiment. That is, all combinations of the various components disclosed in the present invention belong to the scope of the present invention. In addition, the specific description described below may not limit the scope of the present invention.

According to an aspect for achieving the object of the present invention, there is provided a composition for detecting protein-protein interactions comprising: a first construct comprising a polynucleotide encoding a first fusion protein comprising a bait protein and a SEAP first fragment protein; and a second construct comprising a polynucleotide encoding a second fusion protein comprising a prey protein and a SEAP second fragment protein.

In the present invention, the terms “bait protein” and “prey protein” mean proteins interacting with each other, or proteins intended for determining whether the proteins interact with each other. The bait protein and the prey protein may mean materials that interact with each other, such as various therapeutic proteins and signaling proteins. The bait protein and the prey protein may be natural proteins, and may also be domains responsible for functions and parts of natural proteins. In order to detect or screen the interactions, the bait protein may refer to a material known by an experimenter, and the prey protein may refer to an unknown material that is used, but these are not limited thereto. Those skilled in the art may properly select the bait protein and the prey protein by known methods. In the embodiment of the present invention, FKBP12 or FRB may be used as the bait protein or the prey protein.

In the present invention, the terms “SEAP first fragment protein” and “SEAP second fragment protein” mean fragments obtained by cleaving a SEAP full-length protein.

The “SEAP (secreted alkaline phosphatase)” means a form in which a part of a C-terminal of alkaline phosphatase (AP) is deleted. The SEAP may be secreted from cells without a membrane-anchoring domain.

A specific nucleotide sequence of a gene encoding the SEAP and amino acid sequence information of the SEAP may be obtained from a known database such as GenBank of NCBI. However, not only known sequences, but also, as long as they are secreted from cells identically to the SEAP to have alkaline phosphatase activity to allow detection of the protein-protein interactions, homologous proteins or mutant proteins thereof may also be included in the scope of the SEAP provided by the present invention. Specifically, the amino acid sequence of the SEAP may be represented by SEQ ID NO: 1, but is not limited thereto.

The SEAP first fragment protein and the SEAP second fragment protein may be fragments obtained by cleaving a SEAP full-length protein at arbitrary positions. For the purpose of the present invention, the fragment proteins lose the SEAP activity by the cleavage of the full-length protein, but as long as the SEAP activity may be restored by the interaction between the bait protein and the prey protein fused thereto, the cleavage position for the fragment protein is not limited.

The SEAP first fragment protein and the SEAP second fragment protein may be selected from the group consisting of fragments cleaved at amino acid position 8, 60, 372, 379, 387, 404, 418, or 481 from a N-terminal of the SEAP protein. The SEAP first fragment protein and the SEAP second fragment protein may be fragments at the same cleavage position or fragments at different cleavage positions.

Furthermore, a position moved by 8, 7, 6, 5, 4, 3, 2, or 1 amino acid(s) before and after the position may also be a cleavage position for preparing a fragment protein.

Specifically, the SEAP first fragment protein and the SEAP second fragment protein may be selected from the group consisting of fragments cleaved at amino acid positions 1 to 16, 52 to 68, 364 to 395, 396 to 426, or 473 to 489 from the N-terminal of the SEAP protein. The SEAP first fragment protein and the SEAP second fragment protein may be fragments at the same cleavage position or fragments at different cleavage positions.

Even if the SEAP protein and the fragment protein thereof are expressed by specific sequences in the specification, it is apparent that as long as its activity may be maintained, mutant proteins, such as those of substitution, deletion, or addition of unnecessary sequences, are also included in the scope of the present invention.

In the embodiment of the present invention, FKBP12 and FRB are used as the bait protein and the prey protein, a fusion protein obtained by fusing each of various SEAP fragments to the protein (FKBP12 or FRB) is expressed, and then the interaction (binding) between the FKBP12 and the FRB is induced by rapamycin treatment. After that, it is confirmed that some fragment pairs among the various SEAP fragment pairs are complemented with each other to exhibit the SEAP activities. At this time, it is confirmed that the pairs of fragments cleaved at each of amino acid position 8, 60, 372, 379, 387, 404, 418, or 481 from the N-terminal are complemented with each other to exhibit the SEAP activities (see FIGS. 3 and 6).

Specifically, the SEAP first fragment protein and the SEAP second fragment protein may be selected from the group consisting of fragments cleaved at amino acid positions 55 to 68 from the N-terminal of the SEAP protein. The SEAP first fragment protein and the SEAP second fragment protein may be fragments at the same cleavage position or fragments at different cleavage positions.

In the embodiment of the present invention, the FKBP12 and the FRB are used as the bait protein and the prey protein, a fusion protein obtained by fusing each of various fragments cleaved at each of amino acid positions 55 to 68 from the N-terminal to the protein (FKBP12 or FRB) is expressed, and then the interaction (binding) between the FKBP12 and the FRB is induced by rapamycin treatment. After that, it is confirmed that the pairs of fragments are complemented with each other to exhibit the SEAP activities (see FIGS. 4 and 5).

The first construct comprising the polynucleotide encoding the first fusion protein comprising the bait protein and the SEAP first fragment protein and the second construct comprising the polynucleotide encoding the second fusion protein comprising the prey protein and the SEAP second fragment protein may exist in separate vectors or a single vector.

When the constructs exist in the separate vectors, the vector comprising the polynucleotide encoding the first fusion protein comprising the bait protein and the SEAP first fragment protein may be a vector for expressing a protein in which the bait protein is fused to a N-terminal or C-terminal of the SEAP first fragment protein. The vector comprising the polynucleotide encoding the second fusion protein comprising the prey protein and the SEAP second fragment protein may be a vector for expressing a fusion protein in which the prey protein is fused to a N-terminal or C-terminal of the SEAP second fragment protein.

Further, the first construct or the second construct may further include other sequences in addition to the polynucleotide encoding the fusion protein. For example, the other sequence may be a sequence which regulates the expression of the polynucleotide encoding the fusion protein, but is not limited thereto. The polynucleotide and the sequence which regulates the expression of the polynucleotide may be operably linked to each other.

In the present invention, the term “operably linked” means a linked state in which when one polynucleotide fragment links to another polynucleotide fragment, a function or expression thereof is affected by another polynucleotide fragment, but one polynucleotide fragment has no detectable effect on performing the function of another polynucleotide fragment among various possible linking combinations of these polynucleotide fragments. In other words, a polynucleotide sequence encoding a desired protein may be functionally linked to a sequence which regulates the expression of the polynucleotide to perform general functions. Further, in the present invention, “operably linked” may include that the polynucleotide encoding the SEAP fragment protein is linked to the polynucleotide encoding the bait protein or the prey protein to perform the expression or function of the SEAP fragment protein, but is not limited thereto. The operable linkage may be produced using a gene recombination technique well known in the art, and site-specific DNA cleavage and linkage may use enzymes and the like which are generally known in the art.

In the present invention, the term “vector” is an expression vector capable of expressing a desired protein in a suitable host cell and refers to a gene construct including a required regulatory element which is operably linked so that a gene is expressed. The vector of the present invention includes a signal sequence or a leader sequence for membrane targeting or secretion in addition to expression regulatory elements such as a promoter, an operator, an initiation codon, a termination codon, a polyadenylation signal, and an enhancer, and may be variously prepared according to purpose. The promoter of the vector may be constitutive or inducible. Further, the expression vector includes a selective marker for selecting a host cell containing a vector, and a replicable expression vector includes a replication origin. The vector may be self-replicated or integrated with the host DNA. The vector includes a plasmid vector, a cosmid vector, or a viral vector, etc. For the purpose of the present invention, the vector may further comprise an element capable of detecting protein-protein interactions.

According to an aspect for achieving the object of the present invention, the present invention provides a method for detecting protein-protein interactions comprising: (a) introducing to cells a first construct comprising a polynucleotide encoding a first fusion protein comprising a bait protein and a secreted alkaline phosphatase (SEAP) first fragment protein; and a second construct comprising a polynucleotide encoding a second fusion protein comprising a prey protein and a SEAP second fragment protein; (b) expressing the fusion proteins and inducing the protein-protein interactions; and (c) measuring SEAP activities before and after inducing the interactions.

The bait protein, the prey protein, the SEAP first fragment protein, the SEAP second fragment protein, the first construct, and the second construct are as described above.

In the present invention, the term “introduction” means introducing foreign DNA to a cell by transformation or transduction.

The transformation may be performed by various methods known in the art, such as a CaCl₂) precipitation method; the Hanahan method, wherein efficiency is increased by using a reduced material, dimethyl sulfoxide (DMSO), in the CaCl₂ method; an electroporation method, a calcium phosphate precipitation method; a protoplast fusion method; a stirring method using silicon carbide fiber; an agrobacterium-mediated transformation method; a transformation method using PEG; a transformation method using PEI; dextran sulfate, lipofectamine, and drying/inhibition-mediated transformation methods; etc. The transduction means transferring a gene into cells using a virus or viral vector particle by means of infection.

In the present invention, the term “protein expression” means expression of information on foreign DNA introduced into the cells into proteins. The expression may be constitutive or inducible according to a type of promoter. The expression method may use conventional methods generally known in the art.

In the present invention, the term “induction of protein-protein interactions” may mean that the proteins may interact with each other using a specific condition or a specific material. In addition, the interaction may be induced at the same time as the expression of the protein or after the expression of the protein. The method for inducing the protein-protein interactions may be properly selected by known methods according to a type of protein. In the embodiment of the present invention, the FKBP12 and the FRB used as the bait protein and the prey protein are treated with rapamycin to induce the interactions between the proteins.

In the present invention, the term “measurement of the SEAP activity” means measuring the activity of the SEAP as phosphatase. The activity of the phosphatase may be measured by various methods, but specifically, a substrate of the enzyme may be used. In the embodiment of the present invention, the activity of the SEAP was measured using p-nitrophenylphosphate (pNpp) as a substrate and measuring the absorbance at 405 nm, using a property in which a product generated when the pNpp reacts with the SEAP absorbs light at 405 nm.

The method for detecting the protein-protein interactions may further comprise (d) determining that the bait protein and the prey protein interact with each other when the SEAP activity after inducing the interaction measured in step (c) is increased compared to the SEAP activity before inducing the interaction. In the embodiment of the present invention, SEAP activities before/after treatment of rapamycin, which induces the interactions between the FKBP12 and the FRB used as the bait protein and the prey protein, were measured and compared with each other.

Further, the method for detecting the protein-protein interactions may analyze the interactions between the bait protein and the prey protein in a time course. Specifically, since the SEAP of the present invention may be secreted from the cells, the SEAP activity may be measured without destroying the cells to detect the interactions between the bait protein and the prey protein over time.

According to an aspect for achieving the object of the present invention, there is provided a composition for screening a therapeutic agent comprising: a first construct comprising a polynucleotide encoding a first fusion protein comprising a bait protein and a secreted alkaline phosphatase (SEAP) first fragment protein; and a second construct comprising a polynucleotide encoding a second fusion protein comprising a prey protein and a SEAP second fragment protein.

The bait protein, the prey protein, the SEAP first fragment protein, the SEAP second fragment protein, the first construct, and the second construct are as described above.

The “therapeutic agent” means a material for treating diseases which occur due to abnormality of the protein-protein interactions, and specifically, may be a material for restoring the interactions between the bait protein and the prey protein to their original state.

According to an aspect for achieving the object of the present invention, there is provided a composition for screening a promoter or inhibitor for protein-protein interactions comprising: a first construct comprising a polynucleotide encoding a first fusion protein comprising a bait protein and a secreted alkaline phosphatase (SEAP) first fragment protein; and a second construct comprising a polynucleotide encoding a second fusion protein comprising a prey protein and a SEAP second fragment protein.

The bait protein, the prey protein, the SEAP first fragment protein, the SEAP second fragment protein, the first construct, and the second construct are as described above.

The “promoter” or “inhibitor” may be a material which enhances or weakens the interactions between the bait protein and the prey protein.

### Advantageous Effects

According to the composition or the method of the present invention, it is possible to simply detect the protein-protein interactions in the cells without changes in the cell environment (e.g., cell destruction). Furthermore, the composition or the method of the present invention may also be used for detection of materials that enhance or inhibit the protein-protein interactions.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates a secondary structure of a SEAP protein and cleavage positions according to the present invention. Yellow represents an α-helix structure, red represents a β-sheet structure, blue represents a turn structure, and green represents cleavage positions (starting positions of C-terminal fragments). (SEQ ID NO: 1) FIG. 2 is a schematic diagram illustrating vectors comprising a polynucleotide encoding a fusion protein in which a N-terminal fragment and a C-terminal fragment of the SEAP is linked to FKBP and FRB, respectively.

FIG. 3 illustrates results of screening SEAP fragments capable of detecting protein-protein interactions.

FIG. 4 illustrates results of measuring SEAP activities of pairs of SEAP fragments cleaved at each of amino acid positions 55 to 68 from a N-terminal of the SEAP protein.

FIG. 5 illustrates results of measuring SEAP activities of pairs of a N-terminal fragment cleaved at amino acid position 59 from the N-terminal of the SEAP protein and each of C-terminal fragments cleaved at each of amino acid positions 55 to 65 from the N-terminal of the SEAP protein.

FIG. 6 illustrates results of measuring SEAP activities of pairs of SEAP fragments cleaved at different positions.

### MODE FOR INVENTION

Hereinafter, the present invention will be described in more detail with reference to Examples and Experimental Examples. However, these Examples and Experimental Examples are only illustrative of the present invention, and the scope of the present invention is not limited to these Examples and Experimental Examples.

### Example 1: Preparation of Vectors Expressing Fusion Protein Comprising SEAP Fragment

### Example 1-1: Determination of Cleavage Positions of SEAP

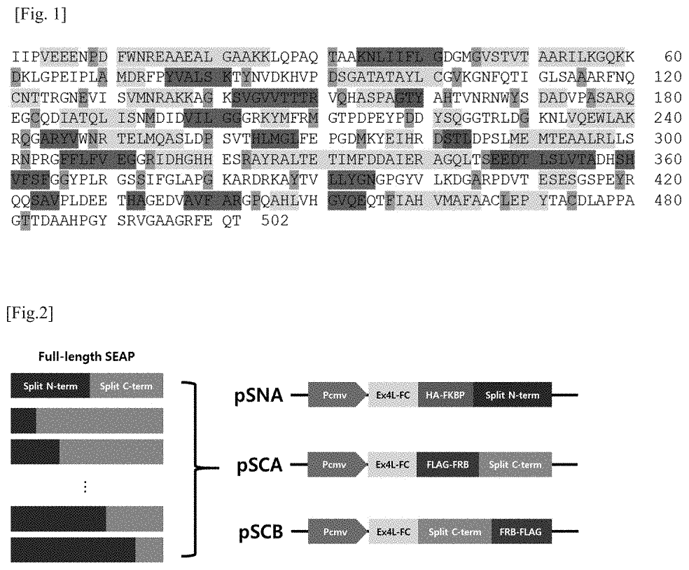

Cleavage positions of SEAP consisting of the amino acid sequence of SEQ ID NO: I were selected from parts where a secondary structure was not confirmed in UniProtKB (ID P05187). In FIG. 1, the secondary structure and the cleavage positions of the SEAP were illustrated. Here, yellow represents an α-helix structure, red represents a β-sheet structure, blue represents a turn structure, and green represents cleavage positions (starting positions of C-terminal fragments).

### Example 1-2: Preparation of Vectors

According to the cleavage position determined in Example 1-1, vectors (FIG. 2) encoding a fusion protein were prepared in which a N-terminal fragment of the SEAP was fused to a C-terminal of FKBP12 and a C-terminal fragment of the SEAP was fused to a N-terminal or C-terminal of FRB.

The FKBP12 and the FRB were known to form a heterodimer mediated with rapamycin.

The vectors used or prepared in Example 1-2 were illustrated in Table 1.

The primers used in Example 1-2 were illustrated in Table 2.

TABLE 1

| Vector | Description | Source |
|---|---|---|
| pAH7 | HA-FKBP expression vector | Cosmogenetech |
| | (P_{hCMV}-NheI-Ex4L-FC-XbaI-HA-FKBP-linker-BamHI-spacer- | (Seoul, Korea) |
| | ApaI-pA_{bGH}) | |
| | P_{hCMV}: Human cytomegalovirus immediate early promoter | |
| | Ex4L: exendin-4 leader sequence | |
| | (MKIILWLCVFGLFLATLFPISWQMPVESGLSSEDSASSES | |
| | FAK (SEQ ID NO: 126)) | |
| | FC: furin cleavage site (RIKR (SEQ ID NO: 127)) | |
| | HA: hemagglutinin tag (YPYDVPDYA (SEQ ID NO: 128)) | |
| | Linker: KGSGSTSGSG (SEQ ID NO: 129); | |
| pAH8 | FRB-FLAG expression vector | Cosmogenetech |
| | (P_{hCMV}-NheI-Ex4L-FC-XbaI-spacer-BamHI-linker-FRB-FLAG- | (Seoul, Korea) |
| | ApaI-pA_{bGH}) | |
| pAH9 | FLAG-FRB expression vector | Cosmogenetech |
| | (P_{hCMV}-NheI-Ex4L-FC-XbaI-FLAG-FRB-linker-BamHI-spacer- | (Seoul, Korea), |
| | ApaI-pA_{bGH}) | |
| pSCA# | FLAG-FRB-scSEAP# expression vector | Example 1 |
| (C-terminal | (P_{hCMV}-NheI-Ex4L-FC-XbaI-FLAG-FRB-linker-BamHI-scSEA | |
| fragment, | P#-ApaI-pA_{bGH}) | |
| #-502) | scSEAP: split C-terminal SEAP fragment | |
| pSCB# | scSEAP#-FRB-FLAG expression vector | Example 1 |
| (C-terminal | (P_{hCMV}-NheI-Ex4L-FC-XbaI-scSEAP#-BamHI-linker-FRB-FL | |
| fragment, | AG-ApaI-pA_{bGH}) | |
| #-502) | | |
| pSNA# | HA-FKBP-snSEAP# expression vector | Example 1 |
| (N-terminal | (P_{hCMV}-NheI-Ex4L-FC-XbaI-HA-FKBP-linker-BamHI-snSEAP | |
| fragment, | #-ApaI-pA_{bGH}). | |
| 1-#) | snSEAP: split N-terminal SEAP fragments | |
| pSEAPX | SEAP expression vector | Genscript |
| | (P_{hCMV}-HindIII-SEAP-EcoRI-pA_{bGH}) | |
| | SEAP was synthesized by removing a BamHI/XbaI restriction | |
| | enzyme cleavage site without a change in amino acids in an | |
| | encoding region (BamHI: changed from ggatcc to gaatcc, | |
| | XbaI: changed from tctaga to tccaga). | |
| | The synthesized SEAP was sub-cloned to pUC57 (pL1). The | |
| | SEAP was cleaved from pL1 using HindIII/EcoRI and inserted | |
| | to pcDNA3.1+ using a corresponding region. | |

TABLE 2

| | | SEQ |
|---|---|---|
| | | ID |
| Primer | Sequence (5′ → 3′) | NO: |
| oSCAR | cagcgggtttaaacgggcccTCATGTCTGCTCGA | 2 |
| | AGCGGCC | |
| oSCA9 | tggaagtggaggatccCCGGACTTCTGGAACCGC | 3 |
| oSCA31 | tggaagtggaggatccACAGCCGCCAAGAACCTC | 4 |
| oSCA45 | tggaagtggaggatccGGGGTGTCTACGGTGACA | 5 |
| oSCA61 | tggaagtggaggatccGACAAACTGGGGCCTGAG | 6 |
| oSCA70 | tggaagtggaggatccGCCATGGACCGCTTCCCA | 7 |
| oSCA83 | tggaagtggaggatccTACAATGTAGACAAACAT | 8 |
| | GTGCC | |
| oSCA91 | tggaagtggaggatccGACAGTGGAGCCACAGCC | 9 |
| oSCA103 | tggaagtggaggatccGTCAAGGGCAACTTCCAG | 10 |
| oSCA115 | tggaagtggaggatccGCCGCCCGCTTTAACCAG | 11 |
| oSCA128 | tggaagtggaggatccGAGGTCATCTCCGTGATG | 12 |
| oSCA140 | tggaagtggaggatccGGGAAGTCAGTGGGAGTG | 13 |
| oSCA152 | tggaagtggaggatccCAGCACGCCTCGCCAGCC | 14 |
| oSCA162 | tggaagtggaggatccCACACGGTGAACCGCAAC | 15 |
| oSCA169 | tggaagtggaggatccTACTCGGACGCCGACGTG | 16 |
| oSCA176 | tggaagtggaggatccGCCTCGGCCCGCCAGGAG | 17 |
| oSCA183 | tggaagtggaggatccTGCCAGGACATCGCTACG | 18 |
| oSCA194 | tggaagtggaggatccATGGACATTGACGTGATCC | 19 |
| oSCA210 | tggaagtggaggatccATGGGAACCCCAGACCCT | 20 |
| oSCA219 | tggaagtggaggatccGATGACTACAGCCAAGGT | 21 |
| oSCA230 | tggaagtggaggatccGGGAAGAATCTGGTGCAG | 22 |
| oSCA242 | tggaagtggaggatccCAGGGTGCCCGGTATGTG | 23 |
| oSCA249 | tggaagtggaggatccAACCGCACTGAGCTCATG | 24 |
| oSCA260 | tggaagtggaggatccCCGTCTGTGACCCATCTC | 25 |
| oSCA274 | tggaagtggaggatccATGAAATACGAGATCCACCG | 26 |
| oSCA281 | tggaagtggaggatccGACTCCACACTGGACCCCT | 27 |
| oSCA302 | tggaagtggaggatccAACCCCCGCGGCTTCTTC | 28 |
| oSCA314 | tggaagtggaggatccCGCATCGACCATGGTCAT | 29 |
| oSCA323 | tggaagtggaggatccAGGGCTTACCGGGCACTG | 30 |
| oSCA346 | tggaagtggaggatccAGCGAGGAGGACACGCTG | 31 |
| oSCA366 | tggaagtggaggatccGGCTACCCCCTGCGAGGG | 32 |
| oSCA373 | tggaagtggaggatccTCCATCTTCGGGCTGGCC | 33 |
| oSCA380 | tggaagtggaggatccGGCAAGGCCCGGGACAGG | 34 |
| oSCA388 | tggaagtggaggatccTACACGGTCTCCTATAC | 35 |
| oSCA405 | tggaagtggaggatccGCCCGGCCGGATGTTACC | 36 |
| oSCA419 | tggaagtggaggatccTATCGGCAGCAGTCAGCA | 37 |
| oSCA444 | tggaagtggaggatccCCGCAGGCGCACCTGGTT | 38 |
| oSCA457 | tggaagtggaggatccTTCATAGCGCACGTCATG | 39 |
| oSCA468 | tggaagtggaggatccTGCCTGGAGCCCTACACC | 40 |
| oSCA474 | tggaagtggaggatccTGCGACCTGGCGCCCCCC | 41 |
| oSCA482 | tggaagtggaggatccACCACCGACGCCGCGCAC | 42 |
| oSCBR | ctgaacctttggatccTGTCTGCTCGAAGCGGCC | 43 |
| oSCB9 | catcaagcgctctagaCCGGACTTCTGGAACCGC | 44 |
| oSCB31 | catcaagcgctctagaACAGCCGCCAAGAACCTC | 45 |
| oSCB45 | catcaagcgctctagaGGGGTGTCTACGGTGACA | 46 |
| oSCB61 | catcaagcgctctagaGACAAACTGGGGCCTGAG | 47 |
| oSCB70 | catcaagcgctctagaGCCATGGACCGCTTCCCA | 48 |
| oSCB83 | catcaagcgctctagaTACAATGTAGACAAACATGT | 49 |
| | GCC | |
| oSCB91 | catcaagcgctctagaGACAGTGGAGCCACAGCC | 50 |
| oSCB103 | catcaagcgctctagaGTCAAGGGCAACTTCCAG | 51 |
| oSCB115 | catcaagcgctctagaGCCGCCCGCTTTAACCAG | 52 |
| oSCB128 | catcaagcgctctagaGAGGTCATCTCCGTGATG | 53 |
| oSCB140 | catcaagcgctctagaGGGAAGTCAGTGGGAGTG | 54 |
| oSCB152 | catcaagcgctctagaCAGCACGCCTCGCCAGCC | 55 |
| oSCB162 | catcaagcgctctagaCACACGGTGAACCGCAAC | 56 |
| oSCB169 | catcaagcgctctagaTACTCGGACGCCGACGTG | 57 |
| oSCB176 | catcaagcgctctagaGCCTCGGCCCGCCAGGAG | 58 |
| oSCB183 | catcaagcgctctagaTGCCAGGACATCGCTACG | 59 |
| oSCB194 | catcaagcgctctagaATGGACATTGACGTGATCC | 60 |
| oSCB210 | catcaagcgctctagaATGGGAACCCCAGACCCT | 61 |
| oSCB219 | catcaagcgctctagaGATGACTACAGCCAAGGT | 62 |
| oSCB230 | catcaagcgctctagaGGGAAGAATCTGGTGCAG | 63 |
| oSCB242 | catcaagcgctctagaCAGGGTGCCCGGTATGTG | 64 |
| oSCB249 | catcaagcgctctagaAACCGCACTGAGCTCATG | 65 |
| oSCB260 | catcaagcgctctagaCCGTCTGTGACCCATCTC | 66 |
| oSCB274 | catcaagcgctctagaATGAAATACGAGATCCACCG | 67 |
| oSCB281 | catcaagcgctctagaGACTCCACACTGGACCCCT | 68 |
| oSCB302 | catcaagcgctctagaAACCCCCGCGGCTTCTTC | 69 |
| oSCB314 | catcaagcgctctagaCGCATCGACCATGGTCAT | 70 |
| oSCB323 | catcaagcgctctagaAGGGCTTACCGGGCACTG | 71 |
| oSCB346 | catcaagcgctctagaAGCGAGGAGGACACGCTG | 72 |
| oSCB366 | catcaagcgctctagaGGCTACCCCCTGCGAGGG | 73 |
| oSCB373 | catcaagcgctctagaTCCATCTTCGGGCTGGCC | 74 |
| oSCB380 | catcaagcgctctagaGGCAAGGCCCGGGACAGG | 75 |
| oSCB388 | catcaagcgctctagaTACACGGTCCTCCTATAC | 76 |
| oSCB405 | catcaagcgctctagaGCCCGGCCGGATGTTACC | 77 |
| oSCB419 | catcaagcgctctagaTATCGGCAGCAGTCAGCA | 78 |
| oSCB444 | catcaagcgctctagaCCGCAGGCGCACCTGGTT | 79 |
| oSCB457 | catcaagcgctctagaTTCATAGCGCACGTCATG | 80 |
| oSCB468 | catcaagcgctctagaTGCCTGGAGCCCTACACC | 81 |
| oSCB474 | catcaagcgctctagaTGCGACCTGGCGCCCCCC | 82 |
| oSCB482 | catcaagcgctctagaACCACCGACGCCGCGCAC | 83 |
| oSNAF | tggaagtggaggatccATCATCCCAGTTGAGGAG | 84 |
| oSNA8a | gatccATCATCCCAGTTGAGGAGGAGAACTGAggg | 85 |
| | cc | |
| oSNA8b | cTCAGTTCTCCTCCTCAACTGGGATGATg | 86 |
| oSNA30 | cagcgggtttaaacgggcccTCACTGTGCAGGCTGC | 87 |
| | AGCTT | |
| oSNA44 | cagcgggtttaaacgggcccTCACATCCCATCGCCC | 88 |
| | AGGAA | |
| oSNA60 | cagcgggtttaaacgggcccTCACTTCTTCTGCCCT | 89 |
| | TTCAG | |
| oSNA69 | cagcgggtttaaacgggcccTCACAGGGGTATCTCA | 90 |
| | GGCCC | |
| oSNA82 | cagcgggtttaaacgggcccTCATGTCTTGGACAGA | 91 |
| | GCCAC | |
| oSNA90 | cagcgggtttaaacgggcccTCATGGCACATGTTTG | 92 |
| | TCTAC | |
| oSNA102 | cagcgggtttaaacgggcccTCACCCGCACAGGTAG | 93 |
| | GCCGT | |
| oSNA113 | cagcgggtttaaacgggcccTCATGCACTCAAGCCA | 94 |
| | ATGGT | |
| oSNA127 | cagcgggtttaaacgggcccTCAGTTGCCGCGTGTC | 95 |
| | GTGTT | |
| oSNA139 | cagcgggtttaaacgggcccTCATGCTTTCTTGGCC | 96 |
| | CGATT | |
| oSNA151 | cagcgggtttaaacgggcccTCACACTCGTGTGGTG | 97 |
| | GTTAC | |
| oSNA161 | cagcgggtttaaacgggcccTCAGGCGTAGGTGCCG | 98 |
| | GCTGG | |
| oSNA168 | cagcgggtttaaacgggcccTCACCAGTTGCGGTTC | 99 |
| | ACCGT | |
| oSNA175 | cagcgggtttaaacgggcccTCAAGGCACGTCGGCG | 100 |
| | TCCGA | |
| oSNA182 | cagcgggtttaaacgggcccTCACCCCTCCTGGCGG | 101 |
| | GCCGA | |
| oSNA193 | cagcgggtttaaacgggcccTCAGTTGGAGATGAGC | 102 |
| | TGCGT | |
| oSNA209 | cagcgggtttaaacgggcccTCAGCGAAACATGTAC | 103 |
| | TTTCG | |
| oSNA218 | cagcgggtttaaacgggcccTCATGGGTACTCAGGG | 104 |
| | TCTGG | |
| oSNA229 | cagcgggtttaaacgggcccTCAGTCCAGCCTGGTC | 105 |
| | CCACC | |
| oSNA241 | cagcgggtttaaacgggcccTCAGCGCTTCGCCAGC | 106 |
| | CATTC | |
| oSNA248 | cagcgggtttaaacgggcccTCACCACACATACCGG | 107 |
| | GCACC | |
| oSNA259 | cagcgggtttaaacgggcccTCAGTCCAGGGAAGCC | 108 |
| | TGCAT | |
| oSNA273 | cagcgggtttaaacgggcccTCAGTCTCCAGGCTCA | 109 |
| | AAGAG | |
| oSNA280 | cagcgggtttaaacgggcccTCATCGGTGGATCTCG | 110 |
| | TATTTC | |
| oSNA301 | cagcgggtttaaacgggcccTCACCTGCTCAGCAGG | 111 |
| | CGCAG | |
| oSNA313 | cagcgggtttaaacgggcccTCAACCACCCTCCACG | 112 |
| | AAGAG | |
| oSNA322 | cagcgggtttaaacgggcccTCAGCTTTCATGATGA | 113 |
| | CCATG | |
| oSNA345 | cagcgggtttaaacgggcccTCAGGTGAGCTGGCCC | 114 |
| | GCCCT | |
| oSNA365 | cagcgggtttaaacgggcccTCATCCGAAGGAGAAG | 115 |
| | ACGTG | |
| oSNA372 | cagcgggtttaaacgggcccTCAGCTCCCTCGCAGG | 116 |
| | GGGTA | |
| oSNA379 | cagcgggtttaaacgggcccTCAAGGGGCCAGCCCG | 117 |
| | AAGAT | |
| oSNA387 | cagcgggtttaaacgggcccTCAGGCCTTCCTGTCC | 118 |
| | CGGGC | |
| oSNA404 | cagcgggtttaaacgggcccTCAGCCGTCCTTGAGC | 119 |
| | ACATA | |
| oSNA418 | cagcgggtttaaacgggcccTCACTCGGGGCTCCCG | 120 |
| | CTCTC | |
| oSNA443 | cagcgggtttaaacgggcccTCAGCCGCGCGCGAAC | 121 |
| | ACCGC | |
| oSNA456 | cagcgggtttaaacgggcccTCAGGTCTGCTCCTGC | 122 |
| | ACGCC | |
| oSNA467 | cagcgggtttaaacgggcccTCAGGCGGCGAAGGCC | 123 |
| | ATGAC | |
| oSNA473 | cagcgggtttaaacgggcccTCAGGCGGTGTAGGGC | 124 |
| | TCCAG | |
| oSNA481 | cagcgggtttaaacgggcccTCAGCCGGCGGGGGGC | 125 |
| | GCCAG | |

1) Preparation of pSCA # (C-Terminal Fragment, #-502) Vector

The vector is a vector expressing a FLAG-FRB-SEAP fragment (C-terminal).

scSEAP (C-terminal fragment of SEAP) was amplified by PCR using a pSEAPX vector as a template and oSCA # and oSCAR as primers. The amplified PCR product and a pAH9 vector were cleaved with BamHI and ApaI restriction enzymes, and each cleaved product was ligated.

2) Preparation of pSCB # (C-Terminal Fragment, #-502) Vector

The vector is a vector expressing a SEAP fragment (C-terminal)-FRB-FLAG.

scSEAP (C-terminal fragment of SEAP) was amplified by PCR using a pSEAPX vector as a template and oSCB # and oSCBR as primers. The amplified PCR product and a pAH8 vector were cleaved with XbaI and BamHI restriction enzymes, and each cleaved product was ligated.

3) Preparation of pSNA # (N-Terminal Fragment, 1-#) Vector

The vector is a vector expressing an HA-FKBP-SEAP fragment (N-terminal).

snSEAP (N-terminal fragment of SEAP) was amplified by PCR using a pSEAPX vector as a template and oSNA # and oSNAF as primers. The amplified PCR product and a pAH7 vector were cleaved with BamHI and ApaI restriction enzymes, and each cleaved product was ligated.

The pSNA8 vector was prepared by hybridizing oSNA8a and OSC8b primers and ligating the hybridized primers to the pAH7 cleaved with BamHI and ApaI.

### Example 2: Screening of SEAP Fragments Capable of Detecting Protein-Protein Interactions

### Example 2-1: Cell Culture

HEK-293T (Human embryonic kidney cell, ATCC: CRL-11268) cells were cultured in DMEM (Dulbecco's modified Eagle's media, Gibco, Seoul, South Korea) treated with a 10% (v/v) FBS (HyClone) and 1% (v/v) penicillin/streptomycin solution (HyClone) and cultured at 37° C. in a humidified atmosphere containing 5% CO₂.

### Example 2-2: Screening

HEK-293T cells were seeded at 2×10⁴ cells per well in a 48-well plate and cultured until 24 hours before transformation.

For the transformation, 0.15 μL of PEI (PEI, <20,000 MW, Cat. No. 23966, Polysciences, Inc., Warrington, Pa., USA; stock solution: 4 mg/mL in ddH20, pH 7.2) was mixed with 0.2 pLg of DNA, and the mixture was vortexed for 5 seconds and then incubated for 20 minutes at 25° C. to prepare 40 μL of DNA-PEI mixture per well. At this time, the DNA was prepared by mixing a vector (pSNA series) containing a SEAP N-terminal fragment and a vector (pSCA or pSCB series) containing a SEAP C-terminal fragment.

After 24 hours of the transformation, the culture medium was replaced with DMEM with 100 nM rapamycin or DMEM without rapamycin.

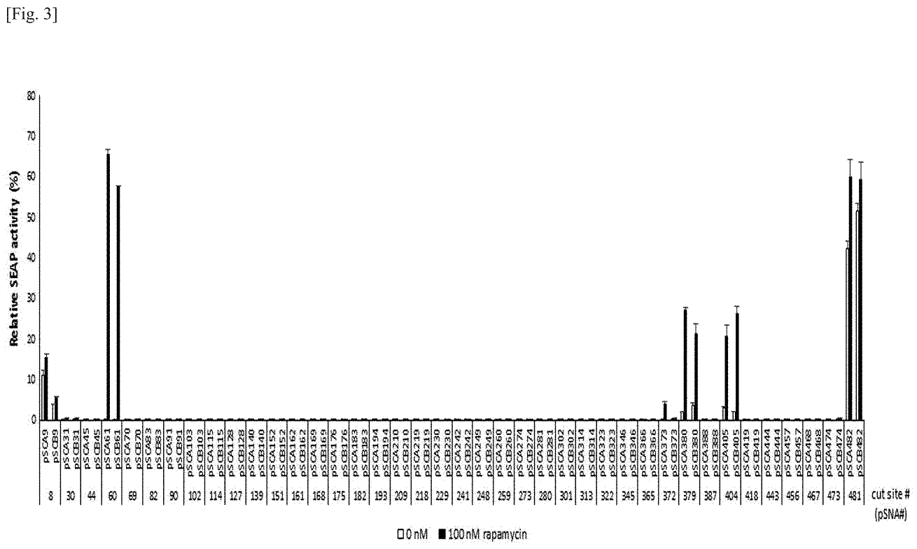

SEAP activity was measured after 24 hours. The SEAP activity was measured in a time course using a p-nitrophenylphosphate (pNpp)-based absorbance (405 nm) measuring method. 80 μL of a culture medium supernatant, 100 μL of a 2× SEAP buffer solution (21% diethanolamine, 20 mM L-homoarginine, and 1 mM MgCl₂, pH 9.8), and 20 μL of 120 mM pNpp were mixed and reacted with one another, and then absorbance at 405 nm was measured. The results were shown in FIG. 3.

Referring to FIG. 3, it can be seen that when an interaction (binding) between FKBP12 and FRB in the cells was induced by treating with rapamycin, some fragments among various SEAP fragments fused to the FKBP12 or FRB were complemented with each other to exhibit SEAP activities.

The SEAP fragment pairs binding to each other to exhibit the SEAP activities are pairs of fragments cleaved at amino acid positions 8, 60, 379, 404, or 481 from the N-terminal (FIG. 3). That is, the pairs of the SEAP fragments cleaved at amino acid positions 8, 60, 379, 404, or 481 from the N-terminal may be used to detect the protein-protein interactions.

### Example 3: Measurement of SEAP Activities of Pairs of SEAP Fragments Cleaved at Each of Amino Acid Positions 55 to 68 from N-Terminal

As the results in the screening of Example 2, it can be seen that the pair of SEAP fragments cleaved at amino acid position 60 from the N-terminal is most excellent in SEAP activity (FIG. 3).

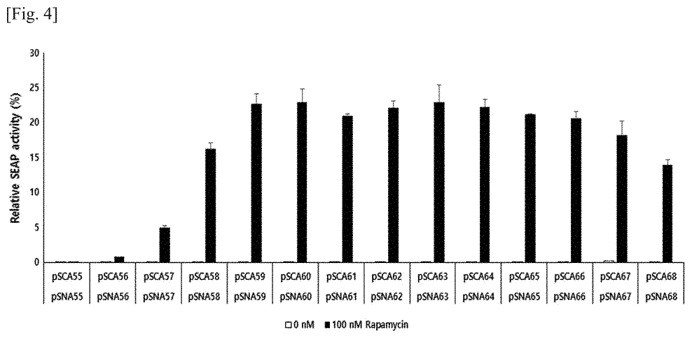

Therefore, SEAP activities of pairs of SEAP fragments cleaved within ±8 from amino acid position 60 were measured by an experiment in the same manner as Example 2, and the results thereof were illustrated in FIG. 4.

Referring to FIG. 4, it can be seen that with the exception of the pair of SEAP fragments cleaved at amino acid position 55 from the N-terminal, all of the fragment pairs were complemented with each other to exhibit excellent SEAP activities. That is, the pairs of the SEAP fragments cleaved at each of amino acid positions 56 to 68 from the N-terminal may be used to detect the protein-protein interactions.

### Example 4: Measurement of SEAP Activities of Pairs of N-Terminal Fragment Cleaved at Amino Acid Position 59 from N-Terminal of SEAP Protein and C-Terminal Fragments Cleaved at Each of Amino Acid Positions 55 to 65 from N-Terminal of SEAP Protein

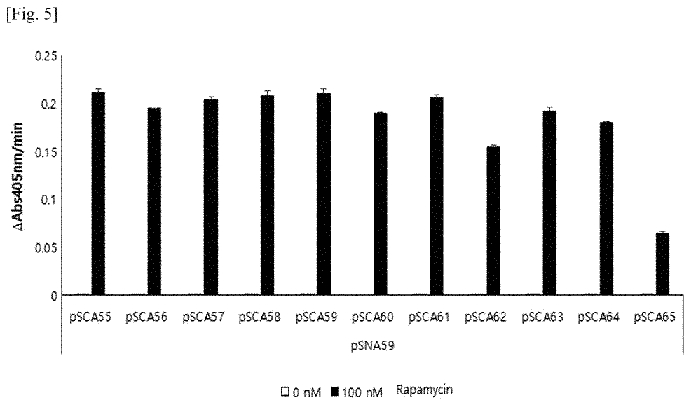

SEAP activities of pairs of a N-terminal fragment pSNA59 cleaved at amino acid position 59 from a N-terminal of a SEAP protein and each of C-terminal fragments pSCA55 to pSCA65 cleaved at each of amino acid positions 55 to 65 from the N-terminal of the SEAP protein were measured by an experiment in the same manner as Example 2, and the results thereof were illustrated in FIG. 5.

Referring to FIG. 5, it can be seen that the N-terminal fragment cleaved at amino acid position 59 from the N-terminal of the SEAP protein and each of the C-terminal fragments cleaved at each of amino acid positions 55 to 65 from the N-terminal of the SEAP protein were complemented with each other to exhibit SEAP activities. That is, the pairs of the N-terminal fragment cleaved at amino acid position 59 from the N-terminal of the SEAP protein and the each of C-terminal fragments cleaved at each of amino acid positions 55 to 65 from the N-terminal of the SEAP protein may be used to detect the protein-protein interactions.

### Example 5: Measurement of SEAP Activities of Pairs of SEAP Fragments Cleaved at Different Positions

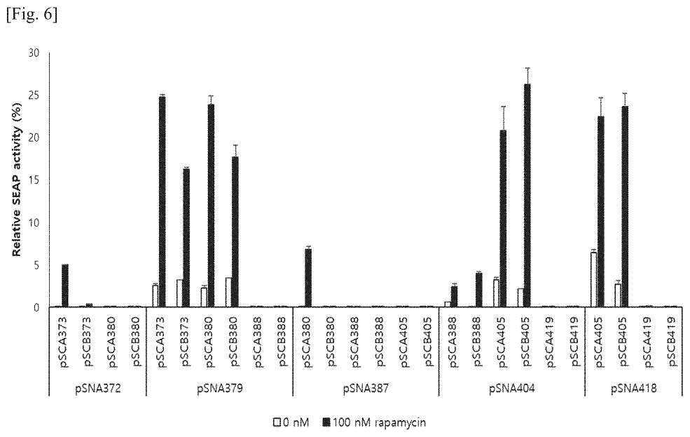

SEAP activities of pairs of SEAP fragments cleaved at different positions were measured by an experiment in the same manner as Example 2, and the results thereof were illustrated in FIG. 6.

Referring to FIG. 6, it can be seen that a SEAP N-terminal fragment pSNA379 cleaved at amino acid position 379 from the N-terminal binds to a SEAP C-terminal fragment pSCA373 or pSCB373 cleaved at amino acid position 372 from the N-terminal to exhibit excellent SEAP activity.

Further, it can be seen that a SEAP N-terminal fragment pSNA387 cleaved at amino acid position 387 from the N-terminal binds to a SEAP C-terminal fragment pSCA380 cleaved at amino acid position 379 from the N-terminal to exhibit excellent SEAP activity.

Further, it can be seen that a SEAP N-terminal fragment pSNA404 cleaved at amino acid position 404 from the N-terminal binds to a SEAP C-terminal fragment pSCA388 or pSCB388 cleaved at amino acid position 387 from the N-terminal to exhibit excellent SEAP activity.

Further, it can be seen that a SEAP N-terminal fragment pSNA418 cleaved at amino acid position 418 from the N-terminal binds to a SEAP C-terminal fragment pSCA405 or pSCB405 cleaved at amino acid position 404 from the N-terminal to exhibit excellent SEAP activity.

That is, the pairs of fragments having different cleavage positions may be used to detect the protein-protein interactions.

It will be appreciated by those skilled in the art that the present invention as described above may be implemented in other specific forms without departing from the technical spirit thereof or essential characteristics. Thus, it is to be appreciated that embodiments described above are intended to be illustrative in every sense, and not restrictive. The scope of the present invention is represented by the claims described below rather than the detailed description, and it is to be interpreted that the meaning and scope of the claims and all changes or modified forms derived from the equivalents thereof come within the scope of the present invention.

1. A composition for detecting protein-protein interactions comprising:
a first construct comprising a polynucleotide encoding a first fusion protein comprising a bait protein and a secreted alkaline phosphatase (SEAP) first fragment protein; and
a second construct comprising a polynucleotide encoding a second fusion protein comprising a prey protein and a SEAP second fragment protein.

2. The composition of claim 1, wherein the SEAP is represented by the amino acid sequence of SEQ ID NO: 1.

3. The composition of claim 1, wherein the SEAP first fragment protein and the SEAP second fragment protein are selected from the group consisting of fragments cleaved at amino acid positions 1 to 16, 52 to 68, 364 to 395, 396 to 426, or 473 to 489 from a N-terminal of the SEAP protein.

4. The composition of claim 1, wherein the SEAP first fragment protein and the SEAP second fragment protein are selected from the group consisting of fragments cleaved at amino acid position 8, 60, 372, 379, 387, 404, 418, or 481 from a N-terminal of the SEAP protein.

5. The composition of claim 1, wherein the SEAP first fragment protein and the SEAP second fragment protein are selected from the group consisting of fragments cleaved at amino acid positions 55 to 68 from a N-terminal of the SEAP protein.

6. A method for detecting protein-protein interactions comprising:
(a) introducing to cells a first construct comprising a polynucleotide encoding a first fusion protein comprising a bait protein and a secreted alkaline phosphatase (SEAP) first fragment protein; and a second construct comprising a polynucleotide encoding a second fusion protein comprising a prey protein and a SEAP second fragment protein;
(b) expressing the fusion proteins and inducing protein-protein interactions; and
(c) measuring SEAP activities before and after inducing the interactions.

7. The method of claim 6, further comprising:
(d) determining that the bait protein and the prey protein interact with each other when the SEAP activity after inducing the interaction measured in step (c) is increased compared to the SEAP activity before inducing the interaction.

8. The method of claim 6, wherein the method analyzes the interactions between the bait protein and the prey protein in a time course.

9. The method of claim 6, wherein the SEAP is represented by the amino acid sequence of SEQ ID NO: 1.

10. The method of claim 6, wherein the SEAP first fragment protein and the SEAP second fragment protein are selected from the group consisting of fragments cleaved at amino acid positions 1 to 16, 52 to 68, 364 to 395, 396 to 426, or 473 to 489 from a N-terminal of the SEAP protein.

11. The method of claim 6, wherein the SEAP first fragment protein and the SEAP second fragment protein are selected from the group consisting of fragments cleaved at amino acid positions 8, 60, 372, 379, 387, 404, 418, or 481 from a N-terminal of the SEAP protein.

12. The method of claim 6, wherein the SEAP first fragment protein and the SEAP second fragment protein are selected from the group consisting of fragments cleaved at amino acid positions 55 to 68 from a N-terminal of the SEAP protein.

13. A composition for screening a therapeutic agent comprising:
a first construct comprising a polynucleotide encoding a first fusion protein comprising a bait protein and a secreted alkaline phosphatase (SEAP) first fragment protein; and
a second construct comprising a polynucleotide encoding a second fusion protein comprising a prey protein and a SEAP second fragment protein.

14. A composition for screening a promoter or inhibitor for protein-protein interactions comprising:
a first construct comprising a polynucleotide encoding a first fusion protein comprising a bait protein and a secreted alkaline phosphatase (SEAP) first fragment protein; and
a second construct comprising a polynucleotide encoding a second fusion protein comprising a prey protein and a SEAP second fragment protein.


SEQUENCE LISTING (129 sequences)

SEQ ID NO: 1 - protein, 502 aa, Unknown
  Secreted Alkaline Phosphatase
IIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYSRVGAAGRFEQT

SEQ ID NO: 2 - DNA, 41 bp, Artificial Sequence
  Synthetic oligonucleotide
cagcgggtttaaacgggccctcatgtctgctcgaagcggcc

SEQ ID NO: 3 - DNA, 34 bp, Artificial Sequence
  Synthetic oligonucleotide
tggaagtggaggatccccggacttctggaaccgc

SEQ ID NO: 4 - DNA, 34 bp, Artificial Sequence
  Synthetic oligonucleotide
tggaagtggaggatccacagccgccaagaacctc

SEQ ID NO: 5 - DNA, 34 bp, Artificial Sequence
  Synthetic oligonucleotide
tggaagtggaggatccggggtgtctacggtgaca

SEQ ID NO: 6 - DNA, 34 bp, Artificial Sequence
  Synthetic oligonucleotide
tggaagtggaggatccgacaaactggggcctgag

SEQ ID NO: 7 - DNA, 34 bp, Artificial Sequence
  Synthetic oligonucleotide
tggaagtggaggatccgccatggaccgcttccca

SEQ ID NO: 8 - DNA, 39 bp, Artificial Sequence
  Synthetic oligonucleotide
tggaagtggaggatcctacaatgtagacaaacatgtgcc

SEQ ID NO: 9 - DNA, 34 bp, Artificial Sequence
  Synthetic oligonucleotide
tggaagtggaggatccgacagtggagccacagcc

SEQ ID NO: 10 - DNA, 34 bp, Artificial Sequence
  Synthetic oligonucleotide
tggaagtggaggatccgtcaagggcaacttccag

SEQ ID NO: 11 - DNA, 34 bp, Artificial Sequence
  Synthetic oligonucleotide
tggaagtggaggatccgccgcccgctttaaccag

SEQ ID NO: 12 - DNA, 34 bp, Artificial Sequence
  Synthetic oligonucleotide
tggaagtggaggatccgaggtcatctccgtgatg

SEQ ID NO: 13 - DNA, 34 bp, Artificial Sequence
  Synthetic oligonucleotide
tggaagtggaggatccgggaagtcagtgggagtg

SEQ ID NO: 14 - DNA, 34 bp, Artificial Sequence
  Synthetic oligonucleotide
tggaagtggaggatcccagcacgcctcgccagcc

SEQ ID NO: 15 - DNA, 34 bp, Artificial Sequence
  Synthetic oligonucleotide
tggaagtggaggatcccacacggtgaaccgcaac

SEQ ID NO: 16 - DNA, 34 bp, Artificial Sequence
  Synthetic oligonucleotide
tggaagtggaggatcctactcggacgccgacgtg

SEQ ID NO: 17 - DNA, 34 bp, Artificial Sequence
  Synthetic oligonucleotide
tggaagtggaggatccgcctcggcccgccaggag

SEQ ID NO: 18 - DNA, 34 bp, Artificial Sequence
  Synthetic oligonucleotide
tggaagtggaggatcctgccaggacatcgctacg

SEQ ID NO: 19 - DNA, 35 bp, Artificial Sequence
  Synthetic oligonucleotide
tggaagtggaggatccatggacattgacgtgatcc

SEQ ID NO: 20 - DNA, 34 bp, Artificial Sequence
  Synthetic oligonucleotide
tggaagtggaggatccatgggaaccccagaccct

SEQ ID NO: 21 - DNA, 34 bp, Artificial Sequence
  Synthetic oligonucleotide
tggaagtggaggatccgatgactacagccaaggt

SEQ ID NO: 22 - DNA, 34 bp, Artificial Sequence
  Synthetic oligonucleotide
tggaagtggaggatccgggaagaatctggtgcag

SEQ ID NO: 23 - DNA, 34 bp, Artificial Sequence
  Synthetic oligonucleotide
tggaagtggaggatcccagggtgcccggtatgtg

SEQ ID NO: 24 - DNA, 34 bp, Artificial Sequence
  Synthetic oligonucleotide
tggaagtggaggatccaaccgcactgagctcatg

SEQ ID NO: 25 - DNA, 34 bp, Artificial Sequence
  Synthetic oligonucleotide
tggaagtggaggatccccgtctgtgacccatctc

SEQ ID NO: 26 - DNA, 36 bp, Artificial Sequence
  Synthetic oligonucleotide
tggaagtggaggatccatgaaatacgagatccaccg

SEQ ID NO: 27 - DNA, 35 bp, Artificial Sequence
  Synthetic oligonucleotide
tggaagtggaggatccgactccacactggacccct

SEQ ID NO: 28 - DNA, 34 bp, Artificial Sequence
  Synthetic oligonucleotide
tggaagtggaggatccaacccccgcggcttcttc

SEQ ID NO: 29 - DNA, 34 bp, Artificial Sequence
  Synthetic oligonucleotide
tggaagtggaggatcccgcatcgaccatggtcat

SEQ ID NO: 30 - DNA, 34 bp, Artificial Sequence
  Synthetic oligonucleotide
tggaagtggaggatccagggcttaccgggcactg

SEQ ID NO: 31 - DNA, 34 bp, Artificial Sequence
  Synthetic oligonucleotide
tggaagtggaggatccagcgaggaggacacgctg

SEQ ID NO: 32 - DNA, 34 bp, Artificial Sequence
  Synthetic oligonucleotide
tggaagtggaggatccggctaccccctgcgaggg

SEQ ID NO: 33 - DNA, 34 bp, Artificial Sequence
  Synthetic oligonucleotide
tggaagtggaggatcctccatcttcgggctggcc

SEQ ID NO: 34 - DNA, 34 bp, Artificial Sequence
  Synthetic oligonucleotide
tggaagtggaggatccggcaaggcccgggacagg

SEQ ID NO: 35 - DNA, 34 bp, Artificial Sequence
  Synthetic oligonucleotide
tggaagtggaggatcctacacggtcctcctatac

SEQ ID NO: 36 - DNA, 34 bp, Artificial Sequence
  Synthetic oligonucleotide
tggaagtggaggatccgcccggccggatgttacc

SEQ ID NO: 37 - DNA, 34 bp, Artificial Sequence
  Synthetic oligonucleotide
tggaagtggaggatcctatcggcagcagtcagca

SEQ ID NO: 38 - DNA, 34 bp, Artificial Sequence
  Synthetic oligonucleotide
tggaagtggaggatccccgcaggcgcacctggtt

SEQ ID NO: 39 - DNA, 34 bp, Artificial Sequence
  Synthetic oligonucleotide
tggaagtggaggatccttcatagcgcacgtcatg

SEQ ID NO: 40 - DNA, 34 bp, Artificial Sequence
  Synthetic oligonucleotide
tggaagtggaggatcctgcctggagccctacacc

SEQ ID NO: 41 - DNA, 34 bp, Artificial Sequence
  Synthetic oligonucleotide
tggaagtggaggatcctgcgacctggcgcccccc

SEQ ID NO: 42 - DNA, 34 bp, Artificial Sequence
  Synthetic oligonucleotide
tggaagtggaggatccaccaccgacgccgcgcac

SEQ ID NO: 43 - DNA, 34 bp, Artificial Sequence
  Synthetic oligonucleotide
ctgaacctttggatcctgtctgctcgaagcggcc

SEQ ID NO: 44 - DNA, 34 bp, Artificial Sequence
  Synthetic oligonucleotide
catcaagcgctctagaccggacttctggaaccgc

SEQ ID NO: 45 - DNA, 34 bp, Artificial Sequence
  Synthetic oligonucleotide
catcaagcgctctagaacagccgccaagaacctc

SEQ ID NO: 46 - DNA, 34 bp, Artificial Sequence
  Synthetic oligonucleotide
catcaagcgctctagaggggtgtctacggtgaca

SEQ ID NO: 47 - DNA, 34 bp, Artificial Sequence
  Synthetic oligonucleotide
catcaagcgctctagagacaaactggggcctgag

SEQ ID NO: 48 - DNA, 34 bp, Artificial Sequence
  Synthetic oligonucleotide
catcaagcgctctagagccatggaccgcttccca

SEQ ID NO: 49 - DNA, 39 bp, Artificial Sequence
  Synthetic oligonucleotide
catcaagcgctctagatacaatgtagacaaacatgtgcc

SEQ ID NO: 50 - DNA, 34 bp, Artificial Sequence
  Synthetic oligonucleotide
catcaagcgctctagagacagtggagccacagcc

SEQ ID NO: 51 - DNA, 34 bp, Artificial Sequence
  Synthetic oligonucleotide
catcaagcgctctagagtcaagggcaacttccag

SEQ ID NO: 52 - DNA, 34 bp, Artificial Sequence
  Synthetic oligonucleotide
catcaagcgctctagagccgcccgctttaaccag

SEQ ID NO: 53 - DNA, 34 bp, Artificial Sequence
  Synthetic oligonucleotide
catcaagcgctctagagaggtcatctccgtgatg

SEQ ID NO: 54 - DNA, 34 bp, Artificial Sequence
  Synthetic oligonucleotide
catcaagcgctctagagggaagtcagtgggagtg

SEQ ID NO: 55 - DNA, 34 bp, Artificial Sequence
  Synthetic oligonucleotide
catcaagcgctctagacagcacgcctcgccagcc

SEQ ID NO: 56 - DNA, 34 bp, Artificial Sequence
  Synthetic oligonucleotide
catcaagcgctctagacacacggtgaaccgcaac

SEQ ID NO: 57 - DNA, 34 bp, Artificial Sequence
  Synthetic oligonucleotide
catcaagcgctctagatactcggacgccgacgtg

SEQ ID NO: 58 - DNA, 34 bp, Artificial Sequence
  Synthetic oligonucleotide
catcaagcgctctagagcctcggcccgccaggag

SEQ ID NO: 59 - DNA, 34 bp, Artificial Sequence
  Synthetic oligonucleotide
catcaagcgctctagatgccaggacatcgctacg

SEQ ID NO: 60 - DNA, 35 bp, Artificial Sequence
  Synthetic oligonucleotide
catcaagcgctctagaatggacattgacgtgatcc

SEQ ID NO: 61 - DNA, 34 bp, Artificial Sequence
  Synthetic oligonucleotide
catcaagcgctctagaatgggaaccccagaccct

SEQ ID NO: 62 - DNA, 34 bp, Artificial Sequence
  Synthetic oligonucleotide
catcaagcgctctagagatgactacagccaaggt

SEQ ID NO: 63 - DNA, 34 bp, Artificial Sequence
  Synthetic oligonucleotide
catcaagcgctctagagggaagaatctggtgcag

SEQ ID NO: 64 - DNA, 34 bp, Artificial Sequence
  Synthetic oligonucleotide
catcaagcgctctagacagggtgcccggtatgtg

SEQ ID NO: 65 - DNA, 34 bp, Artificial Sequence
  Synthetic oligonucleotide
catcaagcgctctagaaaccgcactgagctcatg

SEQ ID NO: 66 - DNA, 34 bp, Artificial Sequence
  Synthetic oligonucleotide
catcaagcgctctagaccgtctgtgacccatctc

SEQ ID NO: 67 - DNA, 36 bp, Artificial Sequence
  Synthetic oligonucleotide
catcaagcgctctagaatgaaatacgagatccaccg

SEQ ID NO: 68 - DNA, 35 bp, Artificial Sequence
  Synthetic oligonucleotide
catcaagcgctctagagactccacactggacccct

SEQ ID NO: 69 - DNA, 34 bp, Artificial Sequence
  Synthetic oligonucleotide
catcaagcgctctagaaacccccgcggcttcttc

SEQ ID NO: 70 - DNA, 34 bp, Artificial Sequence
  Synthetic oligonucleotide
catcaagcgctctagacgcatcgaccatggtcat

SEQ ID NO: 71 - DNA, 34 bp, Artificial Sequence
  Synthetic oligonucleotide
catcaagcgctctagaagggcttaccgggcactg

SEQ ID NO: 72 - DNA, 34 bp, Artificial Sequence
  Synthetic oligonucleotide
catcaagcgctctagaagcgaggaggacacgctg

SEQ ID NO: 73 - DNA, 34 bp, Artificial Sequence
  Synthetic oligonucleotide
catcaagcgctctagaggctaccccctgcgaggg

SEQ ID NO: 74 - DNA, 34 bp, Artificial Sequence
  Synthetic oligonucleotide
catcaagcgctctagatccatcttcgggctggcc

SEQ ID NO: 75 - DNA, 34 bp, Artificial Sequence
  Synthetic oligonucleotide
catcaagcgctctagaggcaaggcccgggacagg

SEQ ID NO: 76 - DNA, 34 bp, Artificial Sequence
  Synthetic oligonucleotide
catcaagcgctctagatacacggtcctcctatac

SEQ ID NO: 77 - DNA, 34 bp, Artificial Sequence
  Synthetic oligonucleotide
catcaagcgctctagagcccggccggatgttacc

SEQ ID NO: 78 - DNA, 34 bp, Artificial Sequence
  Synthetic oligonucleotide
catcaagcgctctagatatcggcagcagtcagca

SEQ ID NO: 79 - DNA, 34 bp, Artificial Sequence
  Synthetic oligonucleotide
catcaagcgctctagaccgcaggcgcacctggtt

SEQ ID NO: 80 - DNA, 34 bp, Artificial Sequence
  Synthetic oligonucleotide
catcaagcgctctagattcatagcgcacgtcatg

SEQ ID NO: 81 - DNA, 34 bp, Artificial Sequence
  Synthetic oligonucleotide
catcaagcgctctagatgcctggagccctacacc

SEQ ID NO: 82 - DNA, 34 bp, Artificial Sequence
  Synthetic oligonucleotide
catcaagcgctctagatgcgacctggcgcccccc

SEQ ID NO: 83 - DNA, 34 bp, Artificial Sequence
  Synthetic oligonucleotide
catcaagcgctctagaaccaccgacgccgcgcac

SEQ ID NO: 84 - DNA, 34 bp, Artificial Sequence
  Synthetic oligonucleotide
tggaagtggaggatccatcatcccagttgaggag

SEQ ID NO: 85 - DNA, 37 bp, Artificial Sequence
  Synthetic oligonucleotide
gatccatcatcccagttgaggaggagaactgagggcc

SEQ ID NO: 86 - DNA, 29 bp, Artificial Sequence
  Synthetic oligonucleotide
ctcagttctcctcctcaactgggatgatg

SEQ ID NO: 87 - DNA, 41 bp, Artificial Sequence
  Synthetic oligonucleotide
cagcgggtttaaacgggccctcactgtgcaggctgcagctt

SEQ ID NO: 88 - DNA, 41 bp, Artificial Sequence
  Synthetic oligonucleotide
cagcgggtttaaacgggccctcacatcccatcgcccaggaa

SEQ ID NO: 89 - DNA, 41 bp, Artificial Sequence
  Synthetic oligonucleotide
cagcgggtttaaacgggccctcacttcttctgccctttcag

SEQ ID NO: 90 - DNA, 41 bp, Artificial Sequence
  Synthetic oligonucleotide
cagcgggtttaaacgggccctcacaggggtatctcaggccc

SEQ ID NO: 91 - DNA, 41 bp, Artificial Sequence
  Synthetic oligonucleotide
cagcgggtttaaacgggccctcatgtcttggacagagccac

SEQ ID NO: 92 - DNA, 41 bp, Artificial Sequence
  Synthetic oligonucleotide
cagcgggtttaaacgggccctcatggcacatgtttgtctac

SEQ ID NO: 93 - DNA, 41 bp, Artificial Sequence
  Synthetic oligonucleotide
cagcgggtttaaacgggccctcacccgcacaggtaggccgt

SEQ ID NO: 94 - DNA, 41 bp, Artificial Sequence
  Synthetic oligonucleotide
cagcgggtttaaacgggccctcatgcactcaagccaatggt

SEQ ID NO: 95 - DNA, 41 bp, Artificial Sequence
  Synthetic oligonucleotide
cagcgggtttaaacgggccctcagttgccgcgtgtcgtgtt

SEQ ID NO: 96 - DNA, 41 bp, Artificial Sequence
  Synthetic oligonucleotide
cagcgggtttaaacgggccctcatgctttcttggcccgatt

SEQ ID NO: 97 - DNA, 41 bp, Artificial Sequence
  Synthetic oligonucleotide
cagcgggtttaaacgggccctcacactcgtgtggtggttac

SEQ ID NO: 98 - DNA, 41 bp, Artificial Sequence
  Synthetic oligonucleotide
cagcgggtttaaacgggccctcaggcgtaggtgccggctgg

SEQ ID NO: 99 - DNA, 41 bp, Artificial Sequence
  Synthetic oligonucleotide
cagcgggtttaaacgggccctcaccagttgcggttcaccgt

SEQ ID NO: 100 - DNA, 41 bp, Artificial Sequence
  Synthetic oligonucleotide
cagcgggtttaaacgggccctcaaggcacgtcggcgtccga

SEQ ID NO: 101 - DNA, 41 bp, Artificial Sequence
  Synthetic oligonucleotide
cagcgggtttaaacgggccctcacccctcctggcgggccga

SEQ ID NO: 102 - DNA, 41 bp, Artificial Sequence
  Synthetic oligonucleotide
cagcgggtttaaacgggccctcagttggagatgagctgcgt

SEQ ID NO: 103 - DNA, 41 bp, Artificial Sequence
  Synthetic oligonucleotide
cagcgggtttaaacgggccctcagcgaaacatgtactttcg

SEQ ID NO: 104 - DNA, 41 bp, Artificial Sequence
  Synthetic oligonucleotide
cagcgggtttaaacgggccctcatgggtactcagggtctgg

SEQ ID NO: 105 - DNA, 41 bp, Artificial Sequence
  Synthetic oligonucleotide
cagcgggtttaaacgggccctcagtccagcctggtcccacc

SEQ ID NO: 106 - DNA, 41 bp, Artificial Sequence
  Synthetic oligonucleotide
cagcgggtttaaacgggccctcagcgcttcgccagccattc

SEQ ID NO: 107 - DNA, 41 bp, Artificial Sequence
  Synthetic oligonucleotide
cagcgggtttaaacgggccctcaccacacataccgggcacc

SEQ ID NO: 108 - DNA, 41 bp, Artificial Sequence
  Synthetic oligonucleotide
cagcgggtttaaacgggccctcagtccagggaagcctgcat

SEQ ID NO: 109 - DNA, 41 bp, Artificial Sequence
  Synthetic oligonucleotide
cagcgggtttaaacgggccctcagtctccaggctcaaagag

SEQ ID NO: 110 - DNA, 42 bp, Artificial Sequence
  Synthetic oligonucleotide
cagcgggtttaaacgggccctcatcggtggatctcgtatttc

SEQ ID NO: 111 - DNA, 41 bp, Artificial Sequence
  Synthetic oligonucleotide
cagcgggtttaaacgggccctcacctgctcagcaggcgcag

SEQ ID NO: 112 - DNA, 41 bp, Artificial Sequence
  Synthetic oligonucleotide
cagcgggtttaaacgggccctcaaccaccctccacgaagag

SEQ ID NO: 113 - DNA, 41 bp, Artificial Sequence
  Synthetic oligonucleotide
cagcgggtttaaacgggccctcagctttcatgatgaccatg

SEQ ID NO: 114 - DNA, 41 bp, Artificial Sequence
  Synthetic oligonucleotide
cagcgggtttaaacgggccctcaggtgagctggcccgccct

SEQ ID NO: 115 - DNA, 41 bp, Artificial Sequence
  Synthetic oligonucleotide
cagcgggtttaaacgggccctcatccgaaggagaagacgtg

SEQ ID NO: 116 - DNA, 41 bp, Artificial Sequence
  Synthetic oligonucleotide
cagcgggtttaaacgggccctcagctccctcgcagggggta

SEQ ID NO: 117 - DNA, 41 bp, Artificial Sequence
  Synthetic oligonucleotide
cagcgggtttaaacgggccctcaaggggccagcccgaagat

SEQ ID NO: 118 - DNA, 41 bp, Artificial Sequence
  Synthetic oligonucleotide
cagcgggtttaaacgggccctcaggccttcctgtcccgggc

SEQ ID NO: 119 - DNA, 41 bp, Artificial Sequence
  Synthetic oligonucleotide
cagcgggtttaaacgggccctcagccgtccttgagcacata

SEQ ID NO: 120 - DNA, 41 bp, Artificial Sequence
  Synthetic oligonucleotide
cagcgggtttaaacgggccctcactcggggctcccgctctc

SEQ ID NO: 121 - DNA, 41 bp, Artificial Sequence
  Synthetic oligonucleotide
cagcgggtttaaacgggccctcagccgcgcgcgaacaccgc

SEQ ID NO: 122 - DNA, 41 bp, Artificial Sequence
  Synthetic oligonucleotide
cagcgggtttaaacgggccctcaggtctgctcctgcacgcc

SEQ ID NO: 123 - DNA, 41 bp, Artificial Sequence
  Synthetic oligonucleotide
cagcgggtttaaacgggccctcaggcggcgaaggccatgac

SEQ ID NO: 124 - DNA, 41 bp, Artificial Sequence
  Synthetic oligonucleotide
cagcgggtttaaacgggccctcaggcggtgtagggctccag

SEQ ID NO: 125 - DNA, 41 bp, Artificial Sequence
  Synthetic oligonucleotide
cagcgggtttaaacgggccctcagccggcggggggcgccag

SEQ ID NO: 126 - protein, 43 aa, Artificial Sequence
  Synthetic amino acid
MKIILWLCVFGLFLATLFPISWQMPVESGLSSEDSASSESFAK

SEQ ID NO: 127 - protein, 4 aa, Artificial Sequence
  Synthetic amino acid
RIKR

SEQ ID NO: 128 - protein, 9 aa, Artificial Sequence
  Synthetic amino acid
YPYDVPDYA

SEQ ID NO: 129 - protein, 10 aa, Artificial Sequence
  Synthetic amino acid
KGSGSTSGSG